(19) United States
(12) Patent Application Publication
(10) Pub. No.: US 2015/0218638 A1
(43) Pub. Date: Aug. 6, 2015

(54) METHODS FOR ASSESSMENT OF PEPTIDE-SPECIFIC IMMUNITY

(71) Applicants: Hitachi Chemical Co. America, Ltd., Cupertino, CA (US); Hitachi Chemical Co., Ltd., Tokyo (JP)

(72) Inventor: Masato Mitsuhashi, Irvine, CA (US)

(21) Appl. No.: 14/425,284

(22) PCT Filed: Aug. 19, 2013
(86) PCT No.: PCT/US2013/055605

§ 371 Date: Mar. 2, 2015

Related U.S. Application Data
(60) Provisional application No. 61/697,591, filed on Sep. 6, 2012.

(51) Int. Cl. C12Q 1/68

(52) U.S. Cl. CPC C12Q 1/6883; C12Q 2600/158; C12Q 2600/112

(57) ABSTRACT

Embodiments of the invention relate generally to methods for assessing the immune response related to a specific antigen or antigens. In several embodiments, the methods described herein are used to enable a recommendation for a particular type of therapy against a particular antigen, such as a foreign infectious agent or cancer cell. In several embodiments, the methods disclosed herein enable the ongoing monitoring of a subject's immune function.

### RELATED CASES

This application claims the benefit of U.S. Provisional Application No. 61/697,591, filed on Sep. 6, 2012, the entire disclosure of which is incorporated by reference herein.

### FIELD OF THE INVENTION

Several embodiments of the present disclosure relates to methods for assessment of the T-cell immune function of a subject. More specifically, several embodiments of the present disclosure relate to the ex vivo assessment of a subject's peptide-specific T-cell immunity and/or monitoring of peptide vaccine therapy being administered to the subject.

### DESCRIPTION OF RELATED ART

The immune system comprises a set of diverse proteins, cells, tissues, and related processes that serve to protect a host from diseases and/or infections by identifying and eliminating or otherwise inhibiting pathogens. To accomplish this, a key function of the immune system is to distinguish foreign cells or pathogens from endogenous cells, e.g., distinguish between “self” and “non-self” In addition, certain cells of the immune system function to identify a pathogen to which the host was previously exposed, thereby improving the response time of the immune system and the outcome for the host.

### SUMMARY

While humoral immunity can be assessed by measuring IgG titers in serum samples from a patient, up until the methods disclosed herein, cellular immunity has had no straightforward diagnostic counterpart. Among the many benefits disclosed herein, an ex vivo diagnostic for cellular immunity directed against a particular antigen allows assessment of the antigen-specific immunity of a subject, thereby allowing a specifically tailored and informed decision to be made for the overall health of the subject (e.g., whether to treat or not, or what treatment is likely to succeed).

There are therefore provided herein methods for the identification of a subject having cellular immunity against a specific antigen, comprising obtaining a first blood sample and a second blood sample from a subject, exposing the first blood sample to a peptide derived from the specific antigen and exposing the second blood sample to the solvent alone, quantifying the level of expression of one or more T-cell function associated markers in the first and the second whole blood samples and identifying the subject as having cellular immunity against the specific antigen when the expression of the one or more T-cell function associated markers is increased in the first sample as compared to the second sample; or identifying the subject as not having cellular immunity against the specific antigen when the expression of the one or more T-cell function associated markers is substantially similar in the first sample as compared to the second sample.

In several embodiments, the blood samples are whole blood samples. In several embodiments the peptide derived from the specific antigen of interest is dissolved in a solvent, in which case the second blood sample is exposed (under identical conditions) to the solvent without the peptide.

In several embodiments, the quantification is performed by a method comprising adding a primer and a reverse transcriptase to RNA isolated from each of the first blood sample and the second blood sample to generate complementary DNA (cDNA), and contacting the cDNA with sense and antisense primers that are specific for one or more T-cell function associated markers a DNA polymerase to generate amplified DNA. In several embodiments, the T-cell function associated markers comprise one or more of CD25, FoxP3, CTLA4, GARP, IL17, arginase, PD-1, PDL1, and granzyme B. Additionally, the markers may include one or more of GMCSF, interferon gamma, TNFSF2, CXCL10, CCL4, IL2, IL4, IL10, CTLA4, CCL2, and CXCL3.

In several embodiments, the method further comprises treating the subject according to the subject's having cellular immunity to a particular antigen (or not).

There is also a provided herein a method of characterizing the peptide-specific T-cell function of a subject, comprising obtaining a first whole blood sample and a second whole blood sample from a subject, exposing the first whole blood sample to a solvent comprising a peptide derived from an antigen, exposing the second whole blood sample to the solvent alone, and quantifying the level of expression of one or more T-cell function associated markers in the first and the second blood samples, wherein a greater level of expression of the one or more T-cell function associated markers in the first whole blood sample as compared to the second whole blood sample indicates that the subject has cellular immunity to the antigen, and wherein a level of expression of the one or more T-cell function associated markers in the first whole blood sample that is not significantly different from the level of expression as compared to the second whole blood sample indicates that the subject lacks cellular immunity to the antigen.

In several embodiments the quantifying is performed by a method comprising adding a primer and a reverse transcriptase to RNA isolated from each of the first whole blood sample and the second whole blood sample to generate complementary DNA (cDNA), and contacting the cDNA with sense and antisense primers that are specific for one or more T-cell function associated markers selected from the group consisting of CD25, FoxP3, CTLA4, GARP, IL17, arginase, PD-1, PDL1, and granzyme B and a DNA polymerase to generate amplified DNA. Additionally, the method optionally further comprises contacting the cDNA with a DNA polymerase and sense and antisense primers that are specific for one or more T-cell function associated markers selected from the group consisting of GMCSF, interferon gamma, TNFSF2, CXCL10, CCL4, IL2, IL4, IL10, CCL2, and CXCL3.

In several embodiments, the method further comprises treating the subject based on the characterization of the subject's peptide-specific T-cell function.

There are also provided methods for determining the likelihood of the efficacy of a peptide-specific therapy comprising obtaining a first and a second blood sample from a subject, exposing the first blood sample to a solvent comprising a peptide antigen against which the peptide-specific therapy is to be directed, exposing the second blood sample to the solvent alone, quantifying the level of expression of one or more T-cell function associated markers associated with either (i) cytotoxic T-cells or cytotoxic T-cell function or (ii) T-reg and/or MDSC or T-reg and/or MDSC function markers in the first and the second blood samples by a method comprising (i) adding a primer and a reverse transcriptase to RNA isolated from each of the first whole blood sample and the second whole blood sample to generate complementary DNA (cDNA), and contacting the cDNA with sense and antisense primers that are specific for one or more T-cell function associated markers selected from the group consisting of CD25, FoxP3, CTLA4, GARP, IL17, arginase, PD-1, PDL1, and granzyme B and a DNA polymerase to generate amplified DNA; and identifying an increased likelihood of efficacy of the peptide-specific therapy when the T-cell function associated markers are associated with cytotoxic T-cells or cytotoxic T-cell function and expression of the T-cell function associated markers is increased in the first sample as compared to the second sample; or identifying an decreased likelihood of efficacy of the peptide-specific therapy when (a) the T-cell function associated markers are associated with T-reg and/or MDSC or T-reg and/or MDSC function and expression of the T-cell function associated markers is increased in the first sample as compared to the second sample, or (b) the T-cell function associated markers are associated with cytotoxic T-cells or cytotoxic T-cell function and the expression of the T-cell function associated markers is substantially similar in the first sample as compared to the second sample.

Additionally provided is a method for monitoring the ongoing efficacy of a vaccine, comprising obtaining a first and a second blood sample from a subject prior to the subject being exposed to an antigen of interest, exposing the first blood sample to a solvent comprising a peptide derived from the antigen of interest, exposing the second blood sample to the solvent alone, quantifying the level of expression of one or more T-cell function associated markers in the first and the second blood samples by a method comprising: (i) adding a primer and a reverse transcriptase to RNA isolated from each of the first whole blood sample and the second whole blood sample to generate complementary DNA (cDNA), and (ii) contacting the cDNA with sense and antisense primers that are specific for one or more T-cell function associated markers selected from the group consisting of CD25, FoxP3, CTLA4, GARP, IL17, arginase, PD-1, PDL1, and granzyme B and a DNA polymerase to generate amplified DNA, obtaining a third and a fourth blood sample from the subject after a vaccine directed against the antigen of interest has been administered to the subject, exposing the third blood sample to the solvent comprising the peptide derived from the antigen of interest, exposing the fourth blood sample to the solvent alone, quantifying the level of expression of one or more T-cell function associated markers in the third and the fourth blood samples by a method comprising: (i) adding a primer and a reverse transcriptase to RNA isolated from each of the first whole blood sample and the second whole blood sample to generate complementary DNA (cDNA), and (ii) contacting the cDNA with sense and antisense primers that are specific for one or more T-cell function associated markers selected from the group consisting of CD25, FoxP3, CTLA4, GARP, IL17, arginase, PD-1, PDL1, and granzyme B and a DNA polymerase to generate amplified DNA, optionally normalizing the level of expression of one or more T-cell function associated markers in the third and the fourth blood samples based on the level of expression of one or more T-cell function associated markers in the first and the second blood samples; and identifying a maintained or an increased efficacy of the vaccine when the expression of the T-cell function associated markers is increased in the third sample as compared to the first sample; or identifying a decreased efficacy of vaccine when the expression of the T-cell function associated markers is reduced in the third sample as compared to the first sample.

Methods are also provided for identifying a biomarker of cellular immunity, comprising exposing a first portion of a blood sample to a solvent comprising a peptide derived from known antigens, exposing a second portion of the blood sample to the solvent alone, quantifying the level of expression of one or more T-cell function associated markers in the first and the second portions by a method comprising (i) adding a primer and a reverse transcriptase to RNA isolated from each of the first whole blood sample and the second whole blood sample to generate complementary DNA (cDNA), and (ii) contacting the cDNA with sense and antisense primers that are specific for one or more T-cell function associated markers selected from the group consisting of CD25, FoxP3, CTLA4, GARP, IL17, arginase, PD-1, PDL1, and granzyme B and a DNA polymerase to generate amplified DNA; and identifying a biomarker of cellular immunity when the expression of a T-cell function associated marker is increased in the first sample as compared to the second sample or when the expression of a T-cell function associated marker is decreased in the first sample as compared to the second sample.

Additionally, there is provided herein a method for determining the likelihood of the efficacy of a peptide-specific therapy comprising, obtaining a first and a second blood sample from a subject, exposing the first blood sample to a solvent comprising a peptide antigen against which the peptide-specific therapy is to be directed, exposing the second blood sample to the solvent alone, quantifying the level of expression of one or more T-cell function associated markers in the first and the second blood samples, wherein the one or more T-cell function associated markers are associated with either (i) cytotoxic T-cells or cytotoxic T-cell function or (ii) T-reg and/or MDSC or T-reg and/or MDSC function; identifying an increased likelihood of efficacy of the peptide-specific therapy when the T-cell function associated markers are associated with cytotoxic T-cells or cytotoxic T-cell function and expression of the T-cell function associated markers is increased in the first sample as compared to the second sample; or identifying an decreased likelihood of efficacy of the peptide-specific therapy when (a) the T-cell function associated markers are associated with T-reg and/or MDSC or T-reg and/or MDSC function and expression of the T-cell function associated markers is increased in the first sample as compared to the second sample, or (b) the T-cell function associated markers are associated with cytotoxic T-cells or cytotoxic T-cell function and the expression of the T-cell function associated markers is substantially similar in the first sample as compared to the second sample. In some embodiments, an increased likelihood of efficacy is observed when certain T-cell function associated markers are decreased in expression. For example, in several embodiments an increased likelihood of efficacy of a peptide-specific therapy is identified when T-cell function associated markers are associated with cytotoxic T-cells or cytotoxic T-cell function and expression of said T-cell function associated markers is decreased in said first sample as compared to said second sample. Similarly, a decreased likelihood of efficacy can be identified, in certain embodiments, when T-cell function associated markers are associated with T-reg and/or MDSC or T-reg and/or MDSC function and expression of said T-cell function associated markers is decreased in said first sample as compared to said second sample, or the T-cell function associated markers are associated with cytotoxic T-cells or cytotoxic T-cell function and the expression of said T-cell function associated markers is substantially similar in said first sample as compared to said second sample.

As used herein, the term “increased” shall be given its ordinary meaning and shall also refer to increases in expression of greater than about 5%, greater than about 10%, greater than about 15%, greater than about 20%, greater than about 25%, greater than about 50%, or more. Likewise, As used herein, the term “decreased” shall be given its ordinary meaning and shall also refer to decreases in expression of greater than about 5%, greater than about 10%, greater than about 15%, greater than about 20%, greater than about 25%, greater than about 50%, or more. In some embodiments, an increase refers to a statistically significant increase in expression (e.g., p<0.05 based on an art-established statistical analysis). In some embodiments, a decrease refers to a statistically significant decrease in expression (e.g., p<0.05 based on an art-established statistical analysis.)

There is also provided, in several embodiments, a method for identifying a peptide-specific therapy effective to treat an autoimmune disorder comprising obtaining a blood sample from the subject at risk for or suffering from an autoimmune disorder, exposing a first portion of the blood sample to a solvent comprising a specific peptide associated with the peptide-specific therapy, exposing a second portion of the blood sample to the solvent alone, quantifying the level of expression of one or more mRNA associated with self-limiting immune function in the first and the second portion of the blood sample, and determining that the peptide-specific therapy is likely to be efficacious when there is a greater level of expression in the first portion of the blood sample as compared to the second portion of the blood sample.

There is provided in several embodiments, a method for monitoring the ongoing efficacy of a vaccine, comprising, obtaining a first and a second blood sample from a subject prior to the subject being exposed to an antigen of interest, exposing the first blood sample to a solvent comprising a peptide derived from the antigen of interest, exposing the second blood sample to the solvent alone, quantifying the level of expression of one or more T-cell function associated markers in the first and the second blood samples, administering to the subject a vaccine directed against the antigen of interest, obtaining a third and a fourth blood sample from the subject after the administering, exposing the third blood sample to the solvent comprising the peptide derived from the antigen of interest, exposing the fourth blood sample to the solvent alone, quantifying the level of expression of one or more T-cell function associated markers in the third and the fourth blood samples, such as by using a method selected from the group consisting of reverse-transcription polymerase chain reaction (RT-PCR), real-time RT-PCR, northern blotting, microarray gene analysis, digital PCR, RNA sequencing, nanoplex hybridization, fluorescence activated cell sorting, ELISA, mass spectrometry, and western blotting, normalizing the level of expression of one or more T-cell function associated markers in the third and the fourth blood samples based on the level of expression of one or more T-cell function associated markers in the first and the second blood samples, and identifying a maintained or an increased efficacy of the vaccine when the expression of the T-cell function associated markers is increased in the third sample as compared to the first sample, or identifying a decreased efficacy of vaccine when the expression of the T-cell function associated markers is reduced in the third sample as compared to the first sample.

In additional embodiments, there is provided a method for identifying a subject having cellular immunity against a specific antigen, comprising, obtaining a first and a second blood sample from a subject, exposing the first blood sample to a solvent comprising a peptide derived from the specific antigen, exposing the second blood sample to the solvent alone, quantifying the level of expression of one or more T-cell function associated markers in the first and the second blood samples, and identifying the subject as having cellular immunity against the specific antigen when the expression of the T-cell function associated markers is increased in the first sample as compared to the second sample, or identifying the subject as not having cellular immunity against the specific antigen when the expression of the T-cell function associated markers is substantially similar in the first sample as compared to the second sample.

Moreover, there is provided a method of characterizing the peptide-specific T-cell function of a subject, comprising, obtaining a first and a second blood sample from a subject, exposing the first blood sample to a solvent comprising a peptide derived from an antigen, exposing the second blood sample to the solvent alone, quantifying the level of expression of one or more T-cell function associated markers in the first and the second blood samples, wherein a greater level of expression of the one or more T-cell function associated markers in the first sample as compared to the second sample indicates that the subject has cellular immunity to the antigen, and wherein a level of expression of the one or more T-cell function associated markers in the first sample that is not significantly different from the level of expression as compared to the second sample indicates that the subject lacks cellular immunity to the antigen.

In several embodiments, the methods provided herein allow for identification of a biomarker of cellular immunity, the methods comprising, exposing a first portion of a blood sample to a solvent comprising a peptide derived from known antigens, exposing a second portion of the blood sample to the solvent alone, quantifying the level of expression of one or more T-cell function associated markers in the first and the second portions, and identifying a biomarker of cellular immunity when the expression of a T-cell function associated marker is increased in the first sample as compared to the second sample.

In several embodiments, the quantification are achieved using methods such as reverse-transcription polymerase chain reaction (RT-PCR), real-time RT-PCR, northern blotting, microarray gene analysis, digital PCR, RNA sequencing, nanoplex hybridization, fluorescence activated cell sorting, ELISA, mass spectrometry, and western blotting. Other methods, such as quantitative imaging techniques, immunohistochemical methods, immunopreciptation and the like may also be used to quantify the markers of T-cell function, depending on the embodiment.

In several embodiments, the peptide-specific T-cell function is related to T-cell activity directed against one or more of a cancerous condition, an autoimmune condition, a viral infection, a bacterial infection, a fungal infection, a yeast infection, infection due to prions, and infections due to parasites. In some embodiments, the one or more T-cell function associated markers is selected from the group consisting of GMCSF, interferon gamma, TNFSF2, CXCL10, CCL4, IL2, IL4, IL10, CTLA4, CCL2, CXCL3, CD25, FoxP3, CTLA4, GARP, IL17, and arginase. Other markers that are associated with accessory immune functions are also quantified, either in addition to or in place of the T-cell function markers, depending on the embodiment. In addition, evaluation of various pathways associated with immune function can also optionally be evaluated according to the methods disclosed herein (e.g., a specific pathway can be evaluated, in whole or in part) rather than a single marker or panel of markers.

In several embodiments, the whole blood samples are untreated prior to the exposure to the solvent, although in several embodiments, the whole blood samples are treated with an anti-coagulant. In several embodiments, the anti-coagulant comprises heparin. Other anti-coagulants (e.g., citrate) can also be used, depending on the embodiment.

In several embodiments, the samples are exposed to the peptides at a temperature that approximates a physiological temperature. For example, in several embodiments, the exposing is performed at a temperature from about 30° C. to about 42° C. In several embodiments the exposing is performed at a temperature of about 37° C. The duration of exposure may vary, depending on the embodiment (for example based on the relative antigenicity of the peptide). In several embodiments, the exposing is performed for an amount of time of less than about 8 hours. In several embodiments, the amount of time is from about 1 to about 4 hours. Longer or shorter durations can be used in other embodiments.

In addition to enabling the determination of the potential efficacy of a peptide therapy, the identification of a peptide-specific therapy for treating autoimmune disorders, monitoring of the ongoing efficacy of a vaccine, identifying a subject having cellular immunity against a specific antigen, characterizing the peptide-specific T-cell function of a subject, and/or identifying a biomarker of cellular immunity, the methods described herein also, depending on the embodiment, allow for one or more of the following: enabling a medical professional to recommend a peptide-based or non-peptide based therapy, enabling recommendations to be made to medical professionals on whether a peptide therapy would be appropriate for a specific patient, enabling advising a specific peptide-based therapy to be undertaken by a subject in need of a therapy, and methods of treating a subject based on the subject's T-cell immune function.

### BRIEF DESCRIPTION OF THE DRAWINGS

FIGS. 1A-1L depict induction of various immune related mRNAs in response to stimulation by a control agent or by a pool of viral peptides.

FIGS. 2A-2I depict the kinetics of mRNA induction by a pool of viral peptides in comparison to phytohemagglutinin (PHA).

### DETAILED DESCRIPTION

Alterations in immune function, whether function is reduced or increased, are a source of a variety of potential health concerns. For example, overactive immune function, in some cases, can lead to autoimmune diseases. In other cases, decreased immune function can result in a propensity for developing infections, being increasingly at risk for certain diseases, and/or development of cancer of various types. As such, knowing the current immune status of a subject could be a very important piece of information in order to maintain a subject's health or treat a subject for a particular ailment.

### Immune Function—General and Peptide Specific

A variety of cell types, proteins, and pathways that are functionally interrelated make up the immune system. The function of the immune system is to protect a host from disease by identifying and then eliminating pathogens and/or undesired cells (e.g., damaged cells or tumor cells). As many of the pathogens and undesired cells that cause infections or diseases are foreign to a host (or endogenous cells that have lost some “self” aspect and gained some “non-self” aspect) a first step in the immune cascade is often identifying particular cells as “non-self.” Endogenous cells are recognized by the expression of Class I Major Histocompatibility Complex (MHC). Those cells without Class I MHC or with reduced levels of expression may be targeted by the immune system as damaged “self” or “non-self” cells. Foreign pathogens are processed by the immune system and antigens derived from the foreign cells are complexed with MHC, thereby enabling other cells in the immune system to later recognize and target cells bearing such foreign antigens.

While the immune system is comprised of many different cell types, white blood cells (WBCs; leukocytes) are one of the key functional classes immune cells. Lymphocytes are a subtype of WBC that are further divided in Natural Killer (NK) cells, T cells and B cells. Natural killer (NK) cells are specialized, cytotoxic lymphocytes target and destroy, among others, tumor cells, virally infected cells, or damaged “self” cells. T cells are involved in cell-mediated immunity (discussed more below) whereas B cells are primarily responsible for humoral immunity (relating to antibodies). T cells are distinguishable from other lymphocyte types by the presence of the T-cell receptor on the cell surface. T cells are capable of inducing the death of infected somatic or tumor cells. Cytokines (e.g., those released due to inflammation or infection) or presentation of a foreign antigens activate NK cells and cytotoxic T cells, which then release small granules containing various proteins and proteases. One such released protein, perforin, induces pore formation in the membrane of a targeted cell, allowing proteases, such as granzymes, to enter the targeted cell and induce programmed cell death (apoptosis). Thus, T cells, among other immune cell types, play an important role in the ongoing immune function and overall health of a subject.

As mentioned above, T cells express T cell receptors on their surface, which function to recognize specific self MHC molecules expressed on the surface of neighboring cells. Antigen Presenting Cells (APC) work in conjunction with MHC and T cells to combat infection or foreign bodies. APCs process foreign antigens (for example, by phagocytosis and subsequent digestion) and present peptide fragments of the foreign antigens in a complex with the MHC molecules on the surface of the subject's own cells. Peptide-MHC complexes on APCs then interact with the T-cell receptor on certain T cells (e.g., CD4 positive T cells), which is the first step in the establishment of peptide-specific immunity. The fraction of T cells which interact with the APC then produce specific clones comprising pools of effector T cells and memory T cells.

Effector T cells (such as CD8+T cells) are outfitted to specifically recognize the particular foreign antigen that was processed by the APC. They function in the short to mid-term to attack cells expressing the foreign antigen, such as cancers, infected cells, and the like. This is known as the primary cell-mediated immune response.

Memory T cells play a more prominent role in the secondary cell-mediated immune response. The memory cells represent a “pool” of cells that are primed to recognize the particular foreign antigen that was initially presented to the T cells in the form of the peptide-MHC complex. Upon a subsequent exposure to the foreign antigen, the memory T cells can rapidly generate additional effector T cells to combat the cells expressing the foreign antigen.

As a result of the cascades of events outlined above, a subject generates a first, slower response to an antigen (primary cell-mediated immune response), and simultaneously primes their immune system to be prepared to mount a more rapid attack upon a subsequent exposure (secondary cell-mediated immune response).

### Categories of Immune Function

Generally speaking, the immune cascades described above can be characterized by the various types of immune function involved. The main categories of immune activity come together functionally to ensure that the immune system can effectively pilot immune cells to an area of the body where they are needed and, once there, act to inhibit and/or kill foreign cells or otherwise assist in mounting an immune response. These categories include, but are not limited to, recruitment function, killer function, suppressor (of killer) function, and helper function. A variety of other functions, e.g., antigen presentation, regulation of angiogenesis, pain modulation, etc. are also included.

A threshold step in the initiation of effective immune function is the delivery of immune cells from regions of storage to the site of a foreign cell or antigen. This recruitment function is essential for the proper function of the immune system. Regions from which immune cells are mobilized include, but are not limited to, whole blood, bone marrow, the lymphatic system, and other areas. Recruitment of immune cells allows recognition of foreign antigens at the location of the foreign antigen (e.g., a tumor or infection). Recruitment is often initiated by release of chemokines from foreign cells or even from endogenous cells that are in the region of the foreign cell. Recruitment function that is compromised or malfunctioning means that immune cells cannot be properly instructed on where to go to function. Recruiter function is provided, in some embodiments, by chemokines or other chemotactic molecules. In some embodiments, chemokines of a particular motif function to recruit other immune molecules. For example, in several embodiments, CCL molecules, such as CCL-2, CCL-4, CCL-8, or CCL-20 are involved in recruiting other immune cells. In other embodiments, CXCL molecules, such as CXCL-3 or CXCL-10 are involved. In some embodiments, other chemokine effectors, whether C-C or C-X-C motif or another variety, are involved.

After having been recruited to the proper location, the other types of immune cells can perform their designated function, which in some embodiments, is to kill the target cell(s). In some embodiments, the death of the target cells occurs via apoptosis. For example, when the target is a tumor, one or more cells having killer function are recruited to the target site. In some embodiments, such killer cells express one or more of molecules such as Granzyme B, perforin, TNFSF1 (lymphotoxin), TNFSF2 (TNF-alpha), TNFSF 5 (CD40 ligand), TNFSF6 (Fas ligand), TNFSF14 (LIGHT), TNFSF 15 (TL1A), and/or CD16. As such, the recruitment of these cells to the target site initiates a cascade that results in the destruction of the target cells, and thus realizes one goal of the immune system, e.g., destruction and/or removal of a foreign body or cell.

Another function of the immune system, is to provide a negative influence (e.g., a limit) on the killing function of the immune system. This is, at least in part, to prevent overactive immune function, which could lead to autoimmune disorders). Cells that participate is this limiting function can be recognized by markers including, but not limited to, IL10, TGF-beta, (forkhead box p3) FoxP3, CD25, arginase, CTLA-4, and/or PD-1. These cells help to ensure proper overall immune function by keeping the activity of the immune system balanced.

Additional cells types may be involved, to varying degrees, in the killing function of the immune system and/or the self-limiting function of the immune system. Helper T-cells (Th cells) are a sub-group of lymphocytes that assist in maximizing the capabilities of the immune system. Unlike the cells described above, Th cells lack cytotoxic or phagocytic activity. Th cells are, however, involved in activating and directing other immune cells such as the cytotoxic T cells (e.g., the killer cells described above). Th cells are divided into two main subcategories (Th1 or Th2) depending on, among other factors, what cell type they primarily activate, what cytokines they produce, and what type of immune stimulation is promoted. For example, Th1 cells primarily partner with macrophages, while Th2 cells primarily partner with B-cells. Th1 cells produce interferon-gamma, TNF-beta, and IL-2, while Th2 cells product IL1, IL5, IL6, IL10 and IL13. Markers of the subsets of Th cells are known and can be used to identify the induction of certain Th cell subtypes in response to stimulation. For example, the induction of IL2 or IFNG represent responses to stimulation by Th1 cells, while induction of IL4 or IL10 represent responses to stimulation by Th2 cells. Other subtypes, such as Th17 are represented by other markers, such as IL17 (see e.g., Tables 5 and 6).

A variety of other markers of accessory immune functions also exist. For example, antigen presentation function can be evaluated by measurement of GMCSF, B-cell proliferation can be evaluated by measurement of IGH2, angiogenesis can be evaluated by measurement of VEGF (which may be of particular importance with respect to possible tumor formation, as many tumors have increased blood flow demands), pain can be evaluated by measurement of POMC.

The killing function of the immune system such as the function of NK cells and cytotoxic T cells is important, in several embodiments, for destruction of cancerous cells and combating infections and/or inflammation (among other applications). Due to their ability to potentially kill both unwanted target cells as well as normal endogenous cells, NK cells possess two types of surface receptors, activating receptors and inhibitory receptors. Together, these receptors serve to balance the activity of, and therefore regulate, the cytotoxic activity of NK cells. Activating signals are required for activation of NK cells, and may involve cytokines (such as interferons), activation of FcR receptors to target cells against which humoral immune responses have been mounted, and/or foreign ligand binding to various activating NK cell surface receptors. Targeted cells are then destroyed by the apoptotic mechanism described above.

Similarly, cytotoxic T cells also require activation, thought to be through a two signal process resulting in the presentation of a foreign (e.g., non-self) antigen to the cytotoxic T cells. Once activated, cytotoxic T cells undergo clonal expansion, largely in response to interleukin-2 (IL-2), a growth and differentiation factor for T cells. Cytotoxic T cells function somewhat similarly to NK cells in the induction of pore formation and apoptosis in target cells. In several embodiments, the identification of a subject's specific T-cell function is important to determining the ability of the subject to mount a response to a particular foreign antigen. In addition, in several embodiments, the function of the T cells determines, at least in part, the rate of response of the subject's immune function.

The self-limiting nature of immune function is believed to be moderated by T-reg and MDSCs. Developing in the thymus, many T-reg express the forkhead family transcription factor FoxP3 (forkhead box p3). In many disease states, particularly cancers, alterations in T-reg numbers, particularly those T-reg expressing Foxp3, are found. For example, patients with tumors have a local relative excess of Foxp3 positive T cells which inhibits the body's ability to suppress the formation of cancerous cells. MDSCs do not destroy offensive T cells, however, they do alter how cytotoxic T cells behave. MDSCs secrete arginase (ARG), a protease that breaks down the amino acid arginine. Lymphocytes, including cytotoxic T cells and NK cells are indirectly dependent on arginine for activation. Secretion of ARG by MDSCs limits the activation of NK cells and cytotoxic T cells. Thus, in several embodiments, peptide specific immunity may be impacted by the limitation of activation of T cells. In some cases, self-limiting regulation by T-reg and MDSCs may lead to an overall limiting of the functionality of the immune system in a local tissue environment. This has the potential to lead to reduced killing function and which may be insufficient to completely eradicate foreign cells.

As discussed in more detail below, the evaluation of peptide-specific immunity allows assessment of the efficacy of a vaccine, the probability that a subject will (or will not) mount an immune response against a certain antigen, and tracking of immune function related to a specific antigen or class of antigens over time (among other applications). Moreover, by the methods disclosed herein specific antigens (or classes of antigens) can be evaluated with respect to how they stimulate immune function in an individual.

### Diagnostic Measures

A subject may receive immunotherapy, or a vaccination, directed to treat (e.g., eliminate) a particular population of cells in a subject, for example, a cancerous tumor. In response to the immunotherapy or vaccination production of a specific IgG may be induced in the subject. While the titer of that specific IgG can be measured by a variety of immunoassays, these assays are generally not informative with respect to T-cell function that is specific the vaccine. Thus, no routine diagnostic test presently exists to determine the function of T cells directed against specific targets (e.g., a foreign antigen or peptide fragment of that antigen as discussed above). Technical difficulties such as cell isolation, varying culture conditions, and methods to detect or quantify function have precluded such routine diagnostic assays. For example, in order to stimulate the T-cell receptor on a subject's T cells, living cells from that subject are required (T cells do not recognize non-self MHC); in other words, MHC matched donor cells are necessary. This presents an issue with respect to the practical use of diagnostic assays as a subject's own cells must be collected and grown in culture prior to assessing peptide specific T-cell immunity.

To address these limitations and provide a more routine diagnostic assessment of peptide specific T-cell immunity, several embodiments disclosed herein enable use of a panel of one or more exogenous peptides (e.g., those for which an assessment of a subject's immunity is desired). In several embodiments, the exogenous peptides are used to supplement those peptides which have already been processed by the APCs, thereby allowing a more complete determination of the T-cell function of that particular subject.

In several embodiments, the methods disclosed herein are used to monitor the immune function of a subject over time, with respect to a particular peptide target. For example, in some embodiments, a plurality of samples can be collected from the subject and the peptide specific T-cell function is assessed. The results of this monitoring over time, in some embodiments, enable a determination of whether that subject has had or continues to have an increased level of immune activity specific to that peptide. In some embodiments, this monitoring over time can be used to assess whether a subject has developed in immunodeficiency (e.g., congenital or acquired immunodeficiency). In several embodiments, this assessment is made by collecting a sample from the patient and exposing it to a panel of specific peptides. In several embodiments, this exposure will result in induction of certain immune related mRNAs. Subsequent samples collected over time and tested in the same fashion, should an mRNA that was previously induced show a lack of or a diminished induction, would demonstrate a deficient immune response to one or more of the specific peptides in the panel. Advantageously, such a determination enables detection of immunocompromised status in a subject at early stages, thereby allowing appropriate medical intervention, if needed. In some embodiments, rather than a panel of specific peptides, singular peptides are used.

In several embodiments, monitoring of the peptide specific T-cell function can be used to assess the efficacy of a vaccine therapy. Prior to being exposed to an antigen, a subject will not have mRNA induced in response to exposure of their blood samples to a peptide derived from the antigen. If that subject subsequently receives a vaccine comprising that particular antigen, the subject's immune system will process the antigen as described herein. Thereafter, exposure of a blood sample from the subject to a peptide derived from the antigen would induce mRNA (because the subject has generated immune cells that recognize that peptide/antigen). In this manner, the efficacy of a vaccine therapy can be monitored in a subject. For example, after an initial vaccination, the induction of mRNA after exposure to the peptide can be used as a baseline for ongoing monitoring. After collecting future samples and testing them as disclosed herein, a drop in the level of induction over time indicates a loss of efficacy of the vaccine. This suggests, in several embodiments, that a new “booster” of vaccine, or an alternative vaccine, may be necessary. In some embodiments, the determination of induction of mRNA in an initial sample is used as a threshold. In other words, if induction of particular mRNA is not sufficient to reach a certain level, then, in some embodiments, another dose of the vaccine is administered. The testing of the patient's responsiveness is then repeated, and if the threshold induction is met, no additional vaccine administrations need be made (until such time as a “booster” is required, as described above).

In some embodiments, the methods disclosed herein are used to determine whether a subject has been previously exposed to a particular peptide. For example, in several embodiments, a subject had not been previously exposed to a particular antigen, induction of immune related mRNA would likely not be detected. This is due to, at least in part, a relative lack of memory T cells, as discussed above. In contrast, if a subject had in fact been previously exposed to the specific peptide, induction of immune related mRNA would result, as the first exposure would have led to production of a pool of memory T cells. Thus, in several embodiments, a determination can be made of whether the subject is at risk for a hyperactive immune response based on a subsequent exposure to that peptide.

In several embodiments, assessment of a subject's peptide specific immunity enable a determination of whether a subject can mount an effective response against a particular type of foreign cell, e.g., a particular type of cancer. For example, if a specific cancer cell produces a marker (e.g., a peptide) that is unique to the cancer cell (as compared to normal cells) and exposure of a sample from a subject to that specific peptide results in the induction of immune related mRNA associated with the killing function (e.g., cytotoxic T cells), it is likely that the subject is able to mount an immune response against that cancer cell. In contrast, exposure to sample from the subject to the specific peptide of the cancer cell and a lack of induction of immune function related mRNA associated with killing would indicate the subject is less likely to be able to mount an immune response to eliminate the cancer cell. In such instances, adjunct therapy (e.g., surgery, chemical or radiation therapy) may be advisable.

In several embodiments, the methods disclosed herein are used to identify a subject having cellular immunity against a specific antigen and treating that subject accordingly. In several embodiments, such a method comprises obtaining at least two biological samples (e.g., blood samples) from a subject, exposing said one of such samples to a peptide derived from a specific antigen of interest and treating a second sample to identical conditions (without the peptide) and quantifying the level of expression of one or more T-cell function associated markers in the samples. As the expression of the T-cell function markers is analyzed, a subject can be identified as having cellular immunity against the specific antigen when the expression of said one or more T-cell function associated markers is increased in said sample to the peptide as compared to the sample not exposed to the peptide. Likewise, the subject is identified as not having cellular immunity against said specific antigen when the expression of said one or more T-cell function associated markers is substantially similar in the two samples (exposed to peptide vs. not exposed). Based on that identification, the subject can be treated accordingly. Thus, in those embodiments wherein the subject exhibits cellular immunity, an immune-based therapy can be administered to the subject. If no cellular immunity is detected, non-immune based therapies may prove more effective for that subject. In several embodiments, the subject can be “vaccinated” with the peptide from the antigen of interest, in order to boost the cellular immune response that the subject mounts.

In several embodiments, there is also provided a method of treating a subject based on their peptide-specific T-cell function of a subject. Similar to the above, a plurality of blood samples are collected from the subject, at least one of which is exposed to a peptide derived from an antigen of interest and one of which is not so exposed. The level of expression of one or more T-cell function associated markers in the exposed and unexposed samples is quantified and when a greater level of expression of the T-cell function associated markers is present in the exposed sample as compared the non-exposed sample, the subject has cellular immunity to that specific antigen. Conversely, when the level of expression is not significantly different in the exposed versus unexposed samples, the subject lacks cellular immunity to said antigen. Thereafter, administration of a particular therapy is performed; an immune-based therapy if the subject does have cellular immunity and a non-immune based therapy if the subject lacks cellular immunity.

In several embodiments, the quantification is performed according to the methods described herein. For example, in one embodiment, the quantification comprises adding a primer and a reverse transcriptase to RNA isolated from each of samples (exposed and unexposed) to generate complementary DNA (cDNA) and contacting said cDNA with sense and antisense primers that are specific for one or more T-cell function associated markers and a DNA polymerase to generate amplified DNA.

Additionally, several embodiments are directed to determining the likelihood of the efficacy of a peptide-specific therapy and then administering the therapy, if appropriate. In several embodiments, the methods comprise obtaining a first and a second blood sample from a subject, exposing said first blood sample to a solvent comprising a peptide antigen against which said peptide-specific therapy is to be directed and exposing said second blood sample to said solvent alone. Thereafter the level of expression of one or more T-cell function associated markers is quantified. These markers may be, depending on the embodiment, markers of cytotoxic T-cells or cytotoxic T-cell function or T-reg and/or MDSC function markers. A peptide-specific therapy is then identified as having an increased likelihood of efficacy when said T-cell function associated markers are associated with cytotoxic T-cells or cytotoxic T-cell function and expression of said T-cell function associated markers is increased in said first sample as compared to said second sample. Alternatively, the quantification may result in an identification of a decreased likelihood of efficacy of the peptide-specific therapy when (a) said T-cell function associated markers are associated with T-reg and/or MDSC or T-reg and/or MDSC function and expression of said T-cell function associated markers is increased in said first sample as compared to said second sample, or (b) said T-cell function associated markers are associated with cytotoxic T-cells or cytotoxic T-cell function and the expression of said T-cell function associated markers is substantially similar in said first sample as compared to said second sample. Based on the identification of the peptide-specific therapy being effective, the therapy can then either be administered to the subject (when determined likely to be effective) or administration can be foregone (when determined unlikely to be effective). In several embodiments, the peptide-specific therapy is an anti-cancer therapy.

Also, in one embodiment, there is for identifying a peptide-specific therapy effective to treat an autoimmune disorder in a subject and thereafter treating the subject. The method comprises, in several embodiments, obtaining a blood sample from said subject at risk for or suffering from an autoimmune disorder, exposing a first portion of said blood sample to a solvent comprising a specific peptide associated with said peptide-specific therapy, exposing a second portion of said blood sample to said solvent alone, and quantifying the level of expression of one or more mRNA associated with self-limiting immune function in said first and said second portion of said blood sample, determining that the peptide-specific therapy is likely to be efficacious when there is a greater level of expression in the first portion of the blood sample as compared to the second portion of the blood sample, and when the peptide-specific therapy is determined to be likely to be effective, administering the peptide-specific therapy to the subject.

In several embodiments, the methods disclosed herein can be used to determine the potential efficacy of a particular type of peptide vaccine. For example, in certain autoimmune situations, there exist cells or proteins that attack other endogenous cells within a subject's body (as occurs with type I diabetes). Several embodiments of the methods disclosed herein are useful for determining the potential efficacy of a putative peptide vaccine. In other words, if the exposure of a sample from a subject to the putative peptide vaccine results in induction of mRNA related to the self-limiting immune function discussed above, then it is likely that that putative peptide vaccine would be efficacious to treat the autoimmune situation. This is because the diagnostic test has indicated that the peptide will induce a set of cells associated with self-limitation of the subject immune function. Moreover, these cells will be specifically directed against those cells that also bear the specific peptide and are attacking endogenous cells (e.g., the “culprit” cells).

### Target Conditions

In several embodiments, the methods and compositions disclosed here are used to assess a subject's ability to mount an immune response against a variety of different specific antigens. For example, in several embodiments, the foreign antigen can be derived from cancerous cells (or other mutated cells). Markers specific to a variety of cancers can be tested for, depending on the embodiment. For example, in several embodiments a subject can be tested for specific immunity to a variety of cancers including, but not limited to lymphoblastic leukemia (ALL), acute myeloid leukemia (AML), adrenocortical carcinoma, kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma, breast cancer, bronchial tumors, burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma hairy cell leukemia, renal cell cancer, leukemia, oral cancer, liver cancer, lung cancer, lymphoma, melanoma, ocular cancer, ovarian cancer, pancreatic cancer, prostate cancer, pituitary cancer, uterine cancer, and vaginal cancer.

Alternatively, in several embodiments, a subject can be tested for specific immunity to infections cells derived from bacteria, viruses, fungi, and/or parasites. In some embodiments, T cells responsive to infections of bacterial origin (e.g., infectious bacteria is selected the group of genera consisting of Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, and Yersinia, and mutants or combinations thereof) can be identified by several embodiments of the methods disclosed herein.

In some embodiments, the ability of a subject to mount a specific response against infectious agents of a viral origin can be assessed. The viruses can include, but are not limited to adenovirus, Coxsackievirus, Epstein-Barr virus, hepatitis a virus, hepatitis b virus, hepatitis c virus, herpes simplex virus, type 1, herpes simplex virus, type 2, cytomegalovirus, ebola virus, human herpesvirus, type 8, HIV, influenza virus, measles virus, mumps virus, human papillomavirus, parainfluenza virus, poliovirus, rabies virus, respiratory syncytial virus, rubella virus, and varicella-zoster virus, and combinations thereof. Exosomes can be used to treat a wide variety of cell types as well, including but not limited to vascular cells, epithelial cells, interstitial cells, musculature (skeletal, smooth, and/or cardiac), skeletal cells (e.g., bone, cartilage, and connective tissue), nervous cells (e.g., neurons, glial cells, astrocytes, Schwann cells), liver cells, kidney cells, gut cells, lung cells, skin cells or any other cell in the body.

In several embodiments, the methods disclosed herein are useful for the determination of whether a subject can (or has) mount an immune response to cells having altered metabolic function. In some embodiments, cells with a metabolic discrepancy (as compared to normal cells) express specific identifying markers. A subject may mount an immune response against such cells, in an effort to avoid the possibility of adverse effects based on the malfunctioning cell. For example, the metabolic disruption of a cell may cause a cell to be converted from a normal cell to a pre-cancerous cell. Thus, the immune response can eliminate the cell prior to the cell becoming cancerous. In several embodiments, a propensity for autoimmunity can be detected. In several embodiments, the methods disclosed herein can be used to determine if a subject has in fact previously generated a cell with a certain metabolic malfunction. For example, the methods disclosed herein, in some embodiments, allow for the detection of peptides specific to a particular kind of metabolic dysfunction.

### Methods

In several embodiments, the samples used in the claimed methods are whole blood samples. In several embodiments, the blood samples can be heparinized. Once collected, the blood samples are exposed to at least one specific antigen. As discussed above, the antigen can be derived from any of a variety of sources (cancer cells, viruses, bacteria, etc.). In some embodiments, the exposure occurs at a temperature approximating a physiological temperature. In several embodiments, exposure is performed at a temperature ranging from about 30° C. to about 40° C. In several embodiments, the exposure is performed at approximately 37° C. Depending on the embodiment, the duration of the exposure can vary from about one hour to about eight hours. In some embodiments, exposure lasts for about 1 to about 2 hours, about two hours to about three hours, about three hours to about four hours, about four hours to about five hours, about five hours to about six hours, or about six hours to about eight hours. Longer or shorter durations of exposure are also used, depending on the embodiment. In some embodiments single peptides are used, while in other embodiments, a plurality or panel of peptides is used. In several embodiments, the peptides that make up the panel are all derived from a common general source, e.g., all peptides are from a single type of cancer cell. In some embodiments, the peptides making up the panel are derived from different sources, e.g. some peptides from cancer cells and some peptides from infectious agents such as bacteria. The flexibility in designing the panel of peptides allows customization of the determination of peptides specific T-cell function depending on the needs of a particular subject being tested.

In some embodiments, peptides are diluted with non-reactive solvent (e.g. phosphate buffered saline) in order to tailor the amount of induction that is detected, such that a desired degree of signal gain is achieved (e.g., signal-to-noise ratio is sufficient to allow accurate quantification). Thus, in several embodiments the methods comprise exposing a blood sample (e.g., a whole blood sample) to a peptide derived from an antigen of interest, that peptide have been dispersed (e.g., diluted) in a solvent. In several embodiments, the blood sample is a whole blood sample. In several embodiments, no additional antigen presenting cells are added to the sample. Despite the use of a solvent to dilute the peptide in several embodiments, in other embodiments, a solvent is not used (e.g., if a peptide has been dried, such as with a freeze-dried peptide).

In those embodiments in which mRNA levels are determined, erythrocytes and blood components other than leukocytes are optionally removed from the whole blood sample. In other embodiments, whole blood is used without removal or isolation of any particular cell type. In preferred embodiments, the leukocytes are isolated using a device for isolating and amplifying mRNA. Embodiments of this device are described in more detail in U.S. Pat. Nos. 7,745,180, 7,968,288, 7,939,300, 7,981,608, and 8,076,105, each of which is incorporated in its entirety by reference herein.

In brief, certain embodiments of the device comprise a multi-well plate that contains a plurality of sample-delivery wells, a leukocyte-capturing filter underneath the wells, and an mRNA capture zone underneath the filter which contains immobilized oligo(dT). In certain embodiments, the device also contains a vacuum box adapted to receive the filter plate to create a seal between the plate and the box, such that when vacuum pressure is applied, the blood is drawn from the sample-delivery wells across the leukocyte-capturing filter, thereby capturing the leukocytes and allowing non-leukocyte blood components to be removed by washing the filters. In other embodiments, other means of drawing the blood samples through out of the sample wells and through the across the leukocyte-capturing filter, such as centrifugation or positive pressure, are used. In preferred embodiments of the device, leukocytes are captured on a plurality of filter membranes that are layered together. In several embodiments, the captured leukocytes are then lysed with a lysis buffer, thereby releasing mRNA from the captured leukocytes. The mRNA is then hybridized to the oligo(dT)-immobilized in the mRNA capture zone. Further detail regarding the composition of lysis buffers that may be used in several embodiments can be found in U.S. Pat. No. 8,101,344, which is incorporated in its entirety by reference herein. In several embodiments, cDNA is synthesized from oligo(dT)-immobilized mRNA. In preferred embodiments, the cDNA is then amplified using real time PCR with primers specifically designed for amplification of infection-associated markers. In several embodiments, other methods of quantifying mRNA are used, including, but not limited to, northern blotting, 2-dimensional RT-qPCR, RNase protection, and the like. In several embodiments, other measurement endpoints are used, such as, for example, protein levels and/or functional assays.

After the completion of PCR reaction, the various mRNA (as represented by the amount of PCR-amplified cDNA detected) for one or more leukocyte-function-associated markers are quantified. In certain embodiments, quantification is calculated by comparing the amount of mRNA encoding one or more markers to a reference value. In other embodiments, the reference value is expression level of a gene that is not induced by the stimulating agent, e.g., a house-keeping gene. In certain such embodiments, beta-actin is used as the reference value. Numerous other house-keeping genes that are well known in the art may also be used as a reference value. In other embodiments, a house keeping gene is used as a correction factor, such that the ultimate comparison is the induced expression level of one or more leukocyte-function-associated markers as compared to the same marker from a non-induced (control) sample. In still other embodiments, the reference value is zero, such that the quantification of one or more leukocyte-function-associated markers is represented by an absolute number. In several embodiments, two, three, or more leukocyte-function-associated markers are quantified. In several embodiments, the quantification is performed using real-time PCR and the data are expressed in terms of fold increase (versus an appropriate control). In certain embodiments, the level of expression of one or more T-cell function associated markers is quantified using a method selected from the group consisting of reverse-transcription polymerase chain reaction (RT-PCR), real-time RT-PCR, northern blotting, microarray gene analysis, digital PCR, RNA sequencing, nanoplex hybridization, fluorescence activated cell sorting, ELISA, mass spectrometry, and western blotting. In some embodiments, an increased likelihood of efficacy is observed when certain T-cell function associated markers are decreased in expression. For example, in several embodiments an increased likelihood of efficacy of a peptide-specific therapy is identified when T-cell function associated markers are associated with cytotoxic T-cells or cytotoxic T-cell function and expression of said T-cell function associated markers is decreased in said first sample as compared to said second sample. Similarly, a decreased likelihood of efficacy can be identified, in certain embodiments, when T-cell function associated markers are associated with T-reg and/or MDSC or T-reg and/or MDSC function and expression of said T-cell function associated markers is decreased in said first sample as compared to said second sample, or the T-cell function associated markers are associated with cytotoxic T-cells or cytotoxic T-cell function and the expression of said T-cell function associated markers is substantially similar in said first sample as compared to said second sample.

As used herein, the term “increased” shall be given its ordinary meaning and shall also refer to increases in expression of greater than about 5%, greater than about 10%, greater than about 15%, greater than about 20%, greater than about 25%, greater than about 50%, or more. Likewise, As used herein, the term “decreased” shall be given its ordinary meaning and shall also refer to decreases in expression of greater than about 5%, greater than about 10%, greater than about 15%, greater than about 20%, greater than about 25%, greater than about 50%, or more. In some embodiments, an increase refers to a statistically significant increase in expression (e.g., p<0.05 based on an art-established statistical analysis). In some embodiments, a decrease refers to a statistically significant decrease in expression (e.g., p<0.05 based on an art-established statistical analysis.)

### EXAMPLES

A specific embodiment will be described with reference to the following example, which should be regarded in an illustrative rather than a restrictive sense.

### Example 1

### Induction of Immune-Function-Related mRNA in Response to Peptide Exposure

While peptides on MHC are known to be derived from digested proteins in APC, however, the present example evaluates the replacement (or supplementation) of endogenous peptides with exogenous peptides. A commercially available peptide pool (CEF peptide pool; Mabtech, www.mabtech.com) was employed, though as discussed above, single peptide or customized panels of peptides are used. This pool contains 23 different class-I restricted peptides, all defined as common CD8+T-cell epitopes derived from cytomegalovirus, Epstein-Barr virus and influenza virus. This panel induces IFN-γ production by virus-specific CD8+T cells in almost 90% of Caucasians and also elicits Perforin, Granzyme B and MIP-1β responses in many individuals.

The stock peptide (200 μg/mL) was diluted with 1:3, 1:10, 1:10, and 1:100 in PBS, and applied to heparinized whole blood at 37° C. for 4 hours. No additional cells were added. Positive and negative controls leucoagglutinin (PHA-L) and PBS were used, respectively.

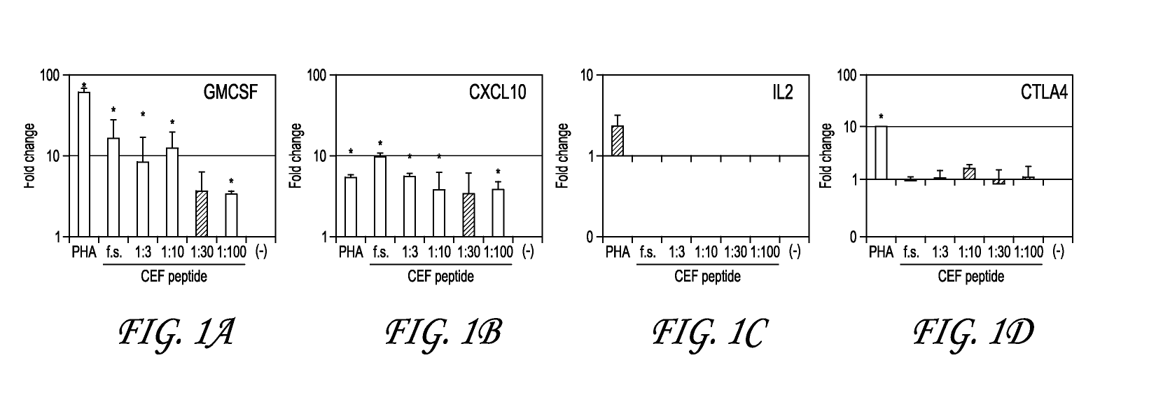

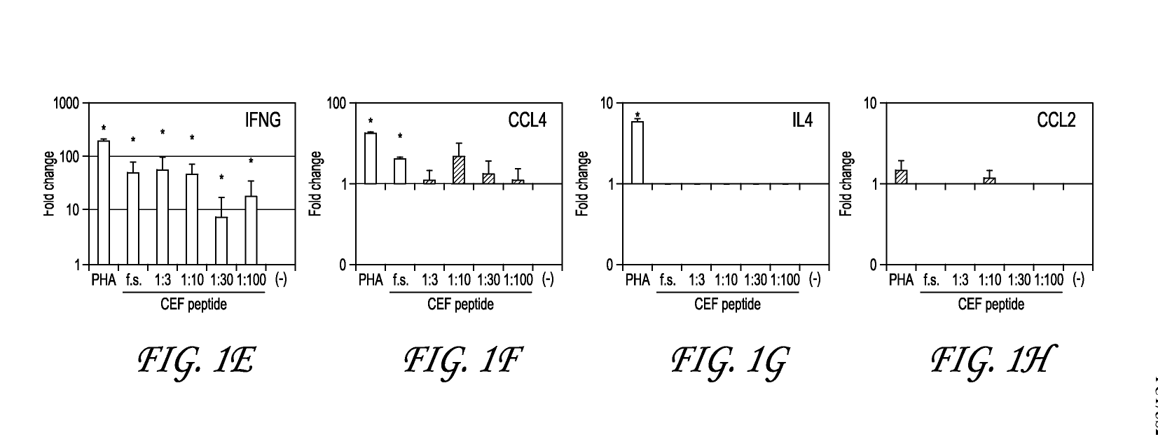

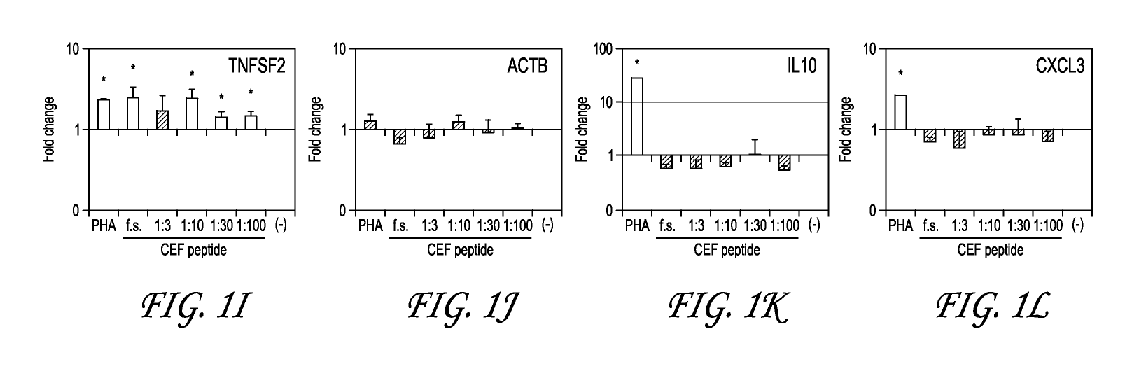

As shown in FIG. 1, positive control PHA-L induced GMCSF, IFNG, TNFSF2, CXCL10, CCL4, IL4, IL10, CTLA4, and CXCL3, whereas control housekeeping gene beta actin (ACTB) was not induced. This confirms the appropriate performance of the. The CEF peptide pool induced GMCSF, IFNG, TNFSF2, CXCL10, and CCL4 in a dose dependent manner.

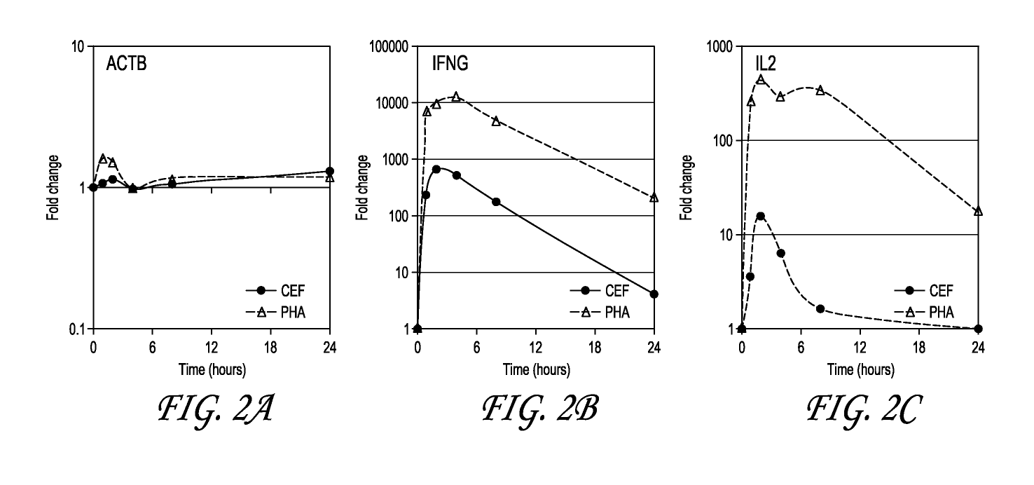

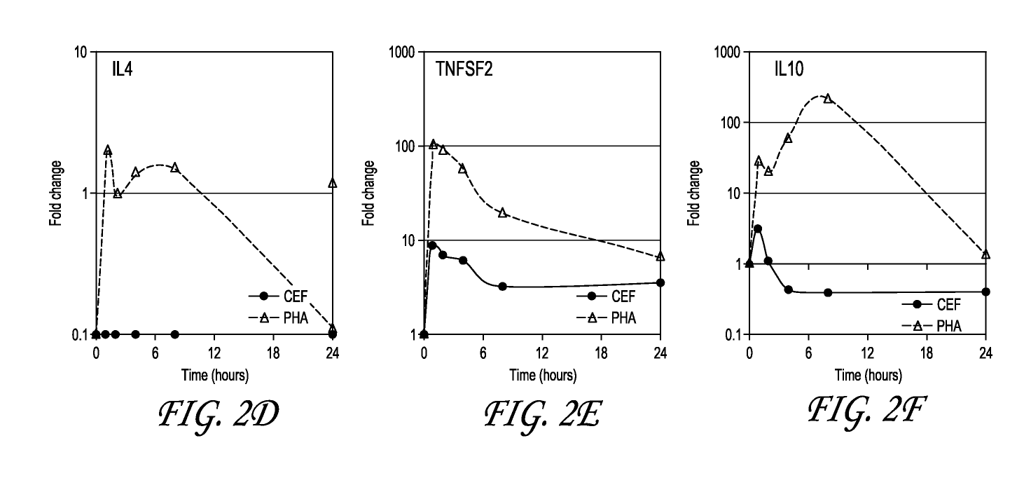

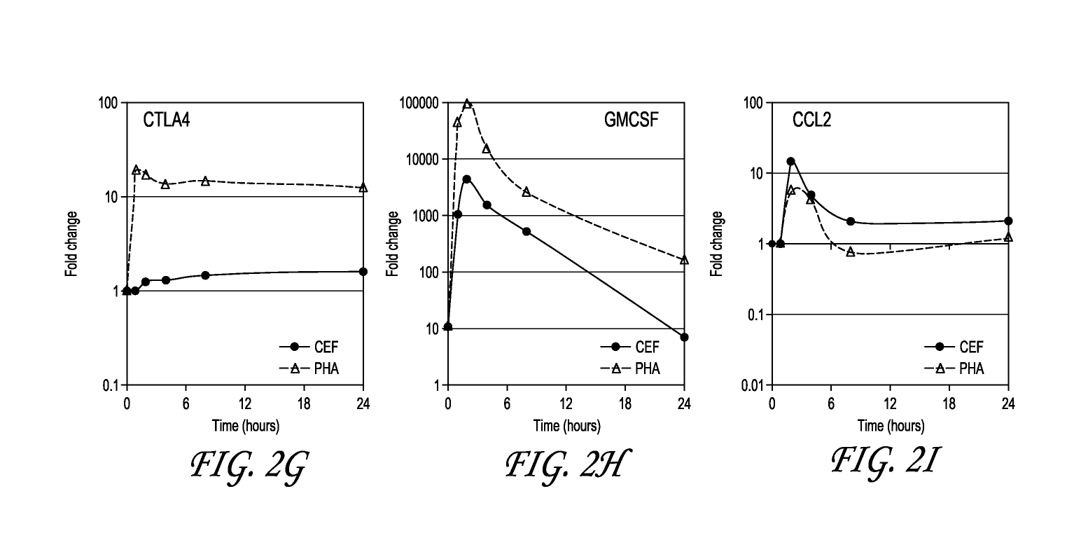

FIG. 2 depicts the kinetics of the induction of mRNA in response to the exposure to the CEF panel. Exposure was performed as described above for durations of 1, 2, 4, 8, and 24 hours and mRNA expression was evaluated by real time PCR (closed circles represent induction by CEF and open triangles are the PBS control). The similarity of the induction of the various mRNA suggest that the exogenous peptides replace (or supplement) existing peptides on MHC, rather than being taken up by cells and processed to be complexed with the MHC (which would shift the kinetic curve for the CEF exposure to the right).

These data indicate that the exposure of leukocytes to exogenous peptides allows for immune-function-related mRNAs to be induced. As such, this experiment demonstrates that peptide specific T cell immunity can be assessed by the ex vivo methods disclosed herein.

1. A method for treating a subject suffering from cancer, comprising:
(A) having a first whole blood sample and a second whole blood sample from a subject sent to a laboratory for said laboratory to perform an assay comprising the following steps:
(1) exposing said first whole blood sample to a solvent comprising a peptide derived from said specific antigen;
(2) exposing said second whole blood sample to said solvent alone;
(3) quantifying the level of expression of one or more T-cell function associated markers in said first and said second whole blood samples by a method comprising:
(i) adding a primer and a reverse transcriptase to RNA isolated from each of the first whole blood sample and the second whole blood sample to generate complementary DNA (cDNA), and
(ii) contacting said cDNA with sense and antisense primers that are specific for one or more T-cell function associated markers selected from the group consisting of CD25, FoxP3, CTLA4, GARP, IL17, arginase, PD-1, PDL1, and granzyme B and a DNA polymerase to generate amplified DNA, and
(iii) measuring said amplified DNA to determine said level of expression of said one or more T-cell function associated markers, wherein an increase in said level of expression in said first sample as compared to said second sample indicates that said subject has cellular immunity against a specific antigen; and
(B) treating said subject suffering from cancer with an immune-based therapy when said subject has cellular immunity against a specific antigen.

2.-53. (canceled)

54. The method of claim 1, wherein the immune-based therapy is a peptide-based therapy.

55. The method of claim 1, further comprising contacting said cDNA with a DNA polymerase and sense and antisense primers that are specific for one or more T-cell function associated markers selected from the group consisting of GMCSF, interferon gamma, TNFSF2, CXCL10, CCL4, IL2, IL4, IL10, CTLA4, CCL2, and CXCL3.

56. The method of claim 1, wherein the whole blood samples are treated with an anti-coagulant.

57. The method of claim 56, wherein the anti-coagulant comprises heparin.

58. The method of claim 1, wherein the exposing is performed at a temperature from about 30° C. to about 42° C.

59. The method of claim 58, wherein the exposing is performed at a temperature of about 37° C.

60. The method of claim 1, wherein the exposing is performed for an amount of time of less than about 8 hours.

61. The method of claim 60, wherein said amount of time is from about 1 to about 4 hours.

62. The method of claim 1, wherein said peptide derived from said specific antigen is derived from a source selected from the group consisting of a virus, a bacteria, and a cancer cell.

63. A method for treating a subject suffering from an autoimmune disorder, comprising:
(A) having a blood sample from said subject at risk for or suffering from an autoimmune disorder sent to a laboratory for said laboratory to perform an assay comprising the following steps:
(1) exposing a first portion of said blood sample to a solvent comprising a specific peptide associated with a peptide-specific therapy,
(2) exposing a second portion of said blood sample to said solvent alone,
(3) quantifying the level of expression of one or more mRNA associated with self-limiting immune function in said first and said second portion of said blood sample, such as by using a method selected from the group consisting of reverse-transcription polymerase chain reaction (RT-PCR), real-time RT-PCR, northern blotting, fluorescence activated cell sorting, ELISA, mass spectrometry, and western blotting,
wherein said one or more mRNA associated with self-limiting immune function is selected from the group consisting of CD25, FoxP3, CTLA4, GARP, IL17, arginase, PD-1, PDL1, and granzyme B and a DNA polymerase to generate amplified DNA, and
(4) determining that said peptide-specific therapy is likely to be efficacious when said level of expression is greater in said first portion of said blood sample as compared to said second portion of said blood sample; and
(B) treating said subject suffering from an autoimmune disorder with said peptide-specific therapy.

64. The method of claim 63, further comprising contacting said cDNA with a DNA polymerase and sense and antisense primers that are specific for one or more T-cell function associated markers selected from the group consisting of GMCSF, interferon gamma, TNFSF2, CXCL10, CCL4, IL2, IL4, IL10, CTLA4, CCL2, and CXCL3.

65. The method of claim 63, wherein the whole blood samples are treated with an anti-coagulant.

66. The method of claim 65, wherein the anti-coagulant comprises heparin.

67. The method of claim 63, wherein the exposing is performed for an amount of time of less than about 8 hours.

68. The method of claim 63, wherein said amount of time is from about 1 to about 4 hours.

69. The method of claim 63, wherein said peptide derived from said specific antigen is derived from a source selected from the group consisting of a virus, a bacteria, and a cancer cell.

70. A method for treating a subject based on a determination of the ongoing efficacy of a vaccine, comprising:
(A) having a first and a second blood sample from a subject sent to a laboratory to perform a first assay, wherein said first sample and said second sample are obtained prior to said subject being exposed to an antigen of interest, and wherein said first assay comprises:
(1) exposing said first blood sample to a solvent comprising a peptide derived from said antigen of interest;
(2) exposing said second blood sample to said solvent alone;
(3) quantifying the level of expression of one or more T-cell function associated markers in said first and said second blood samples by a method comprising:
(i) adding a primer and a reverse transcriptase to RNA isolated from each of the first whole blood sample and the second whole blood sample to generate complementary DNA (cDNA),
(ii) contacting said cDNA with sense and antisense primers that are specific for one or more T-cell function associated markers selected from the group consisting of CD25, FoxP3, CTLA4, GARP, IL17, arginase, PD-1, PDL1, and granzyme B and a DNA polymerase to generate amplified DNA, and
(iii) measuring said amplified DNA to determine said level of expression of said one or more T-cell function associated markers; and
(B) having a third and a fourth blood sample from said subject sent to a laboratory to perform a second assay, wherein said third and fourth blood samples are obtained after a vaccine directed against said antigen of interest has been administered to said subject, and wherein said second assay comprises:
(1) exposing said third blood sample to said solvent comprising said peptide derived from said antigen of interest;
(2) exposing said fourth blood sample to said solvent alone;
(3) quantifying the level of expression of one or more T-cell function associated markers in said third and said fourth blood samples by a method comprising:
(i) adding a primer and a reverse transcriptase to RNA isolated from each of the first whole blood sample and the second whole blood sample to generate complementary DNA (cDNA),
(ii) contacting said cDNA with sense and antisense primers that are specific for one or more T-cell function associated markers selected from the group consisting of CD25, FoxP3, CTLA4, GARP, IL17, arginase, PD-1, PDL1, and granzyme B and a DNA polymerase to generate amplified DNA,
(iii) measuring said amplified DNA to determine said level of expression of said one or more T-cell function associated markers, and
(iv) normalizing said level of expression of one or more T-cell function associated markers in said third and said fourth blood samples based on said level of expression of one or more T-cell function associated markers in said first and said second blood samples; and
(4) determining the ongoing efficacy of the vaccine,
wherein a maintained or an increased efficacy of the vaccine is determined when said expression of said T-cell function associated markers is increased in said third sample as compared to said first sample, or
wherein a decreased efficacy of vaccine is determined when the expression of said T-cell function associated markers is reduced in said third sample as compared to said first sample; and
(B) Treating said subject when the vaccine has decreased efficacy.

71. The method of claim 1, further comprising contacting said cDNA with a DNA polymerase and sense and antisense primers that are specific for one or more T-cell function associated markers selected from the group consisting of GMCSF, interferon gamma, TNFSF2, CXCL10, CCL4, IL2, IL4, IL10, CTLA4, CCL2, and CXCL3.

72. The method of claim 71, wherein said specific antigen is associated with one or more of a cancerous condition, a viral infection, a bacterial infection, a fungal infection, a yeast infection, an infection due to prions, and infections due to parasites.

73. The method of claim 71, wherein said peptide derived from said specific antigen is derived from a source selected from the group consisting of a virus, a bacteria, and a cancer cell.

74. The method of claim 71, wherein the exposing is performed for an amount of time of less than about 8 hours.

75. The method of claim 74, wherein said amount of time is from about 1 to about 4 hours.
